(19) United States
(12) Patent Application Publication
(10) Pub. No.: US 2023/0074517 A1
(43) Pub. Date: Mar. 9, 2023

(54) BREATH TRAINING SYSTEM

(71) Applicant: HEALTH DEFENSE, INC., Tucker, GA (US)

(72) Inventors: Gary Steven DANIELS, Tucker, GA (US); Jesse John BROWNE, Kennesaw, GA (US)

(21) Appl. No.: 17/930,358

(22) Filed: Sep. 7, 2022

Related U.S. Application Data
(60) Provisional application No. 63/241,176, filed on Sep. 7, 2021.

(51) Int. Cl. A63B 23/18

(52) U.S. Cl. CPC A63B 23/18; A63B 2220/833; A61M 16/06

(57) ABSTRACT

Apparatus and methods for providing breath training. In illustrative embodiments, an operator creates a pressure, either positive or negative, in the lungs. This pressure is transferred through a mask assembly to a breathwork apparatus, which utilizes pistons moving within cylinder volumes to provide numerical feedback to an operator regarding the operator's breathing. Through breath control exercises, the operator of a breathwork apparatus can develop deeper and slower breathing habits which are more efficient. One embodiment provides a breathwork apparatus comprising an exhalation side; a transfer case; an inhalation side; and, a mask assembly, wherein the exhalation side and inhalation side are joined by transfer case which comprises a plurality of channels, such that at least a first channel of a plurality of channels terminates in an exhalation rate meter, and at least a second channel of the plurality of channels terminates in an inhalation rate meter.

### CROSS-REFERENCE TO RELATED APPLICATION

This application claims benefit of priority from U.S. Provisional Application No. 63/241,176, filed Sep. 7, 2021, the disclosure of which is hereby incorporated by reference herein in its entirety.

### FIELD

The present disclosure relates, in illustrative embodiments, to respiration training systems, apparatus, and devices, and more specifically to respiration apparatus and methods of operation of the same that can be used to train persons to have healthy breathing habits.

### BACKGROUND

Breath control is an essential aspect of many professional and hobbyist disciplines, from athletics to music. Accordingly, exercises drawn to improving breath control can have positive impacts on the performances of activities across multiple disciplines that require the use of the respiratory system. In order to better track improvements in breathing exercises, tools which offer empirical metrics by which to measure aspects of an operator's breathing are needed. Accordingly, there is room in the art for improved devices to train athletes, musicians, and others in data-feedback based breath control methods.

### SUMMARY

According to at least one illustrative embodiment of a breathwork apparatus, the apparatus comprises an exhalation side, a transfer case, an inhalation side, and a mask assembly. In other embodiments the mask assembly is separate from the apparatus itself. The exhalation side and inhalation side may be joined by transfer case which comprises a plurality of channels, such that at least a first channel of a plurality of channels terminates in an exhalation rate meter, and at least a second channel of the plurality of channels terminates in an inhalation rate meter. The exhalation side can comprise an exhalation check valve, and the inhalation side comprise an inhalation check valve. The inhalation side may further include a base, a cylinder floor, a cylinder wall, and a piston. The exhalation side may similarly include a base, a cylinder floor, a cylinder wall, and a piston.

The mask assembly may comprise a mask connection in the form of a hose, and the mask assembly may comprise a particulate filter. The mask assembly may also comprise a nasal seal, and an adapter with a dial selector to adjust a restriction within the adapter.

According to illustrative embodiments, a breath training system comprises a breathwork apparatus and a mask assembly. The breathwork apparatus may have an exhalation side, an inhalation side, and a transfer case between the inhalation side and the exhalation side. The mask assembly can be selectively attachable to the breathwork apparatus. In such embodiments the mask assembly further includes a nasal seal, such that when the mask assembly is detached from the breathwork apparatus, an operator of the mask assembly cannot exhale through the operator's nose.

The mask assembly may in illustrative embodiments include an adapter, the adapter operable to connect the mask assembly to the breathwork apparatus, and alternatively to connect the mask assembly to a device that conditions air. In such embodiments, the operator of the mask assembly inhales through the device that conditions air when the operator inhales through the operator's nose. In illustrative embodiments the mask assembly includes a sensor assembly having sensors such as pressure sensors, fluid velocity sensors, oxygen sensors, carbon dioxide sensors, carbon monoxide sensors, moisture content sensors, and spectrometers. The sensor array may transmit data from at least one sensor in the sensor array to a mobile application. The mobile application of these embodiments can monitor data to calculate a parameter such as volume of operator's inspired and expired breath, rate of operator's inspired and expired breath, air speed of operator's inspired and expired breath, rate of oxygen use by the operator, oxygen saturation levels experienced by the operator, instant heart rate of the operator, heart rate variability of the operator, and minute ventilation.

Illustrative embodiments of a breath training system include a belt device measuring the expansion of the abdomen or rib cage of an operator during operation of the system.

A method for operating a breath training system is provided for by at least one illustrative embodiment of the disclosure. Such methods provide a breathwork apparatus having an exhalation side, a transfer case, and an inhalation side. An operator then generates a fluid flow by creating a vacuum in the operator's lungs which is passed to a hose inlet in the transfer case. This generates a negative pressure in the inhalation side through the transfer case, the negative pressure opening a check valve in the inhalation side, and accordingly applying vacuum to a piston in the inhalation side. The operator then reads a scale to determine the motion of the piston within the inhalation side. In illustrative embodiments, the vacuum produced in the lungs of the operator is transferred through a mask assembly.

One illustrative method provides a method for operating a breath training system comprising (a) providing a breathwork apparatus having an exhalation side, a transfer case, and an inhalation side; (b) generating fluid flow, by vacuum produced in the lungs of an operator, through a hose inlet in the transfer case; (c) generating a negative pressure in the inhalation side through the transfer case, the negative pressure opening a check valve in the inhalation side; (d) applying vacuum to a piston in the inhalation side; and, (e) reading a scale to determine the motion of the piston within the inhalation side.

### BRIEF DESCRIPTION OF THE DRAWINGS

The drawings disclose exemplary embodiments in which like reference characters designate the same or similar parts throughout the figures of which:

FIG. 1 is a perspective view of an embodiment of a breathwork apparatus.

FIG. 2 is a rear view of the breathwork apparatus in the embodiment of FIG. 1.

FIG. 3 is a top view of the breathwork apparatus in the embodiment of FIG. 1.

FIG. 4 is a perspective view of a mask assembly.

FIG. 5 is a rear perspective view of the mask assembly apparatus in the embodiment of FIG. 5.

FIG. 6 is a perspective view of a mask assembly.

### DETAILED DESCRIPTION

Unless otherwise indicated, the drawings are intended to be read (for example, cross-hatching, arrangement of parts, proportion, degree, or the like) together with the specification, and are to be considered a portion of the entire written description of this invention. As used in the following description, the terms “horizontal”, “vertical”, “left”, “right”, “up” and “down”, “upper” and “lower” as well as adjectival and adverbial derivatives thereof (for example, “horizontally”, “upwardly”, or the like), simply refer to the orientation of the illustrated structure as the particular drawing figure faces the reader. Similarly, the terms “inwardly” and “outwardly” generally refer to the orientation of a surface relative to its axis of elongation, or axis of rotation, as appropriate.

According to one exemplary embodiment, a breathwork apparatus 100 comprises a multitude of sub-assemblies, including an exhalation side 200, a transfer case 300, an inhalation side 400, and a mask assembly 500.

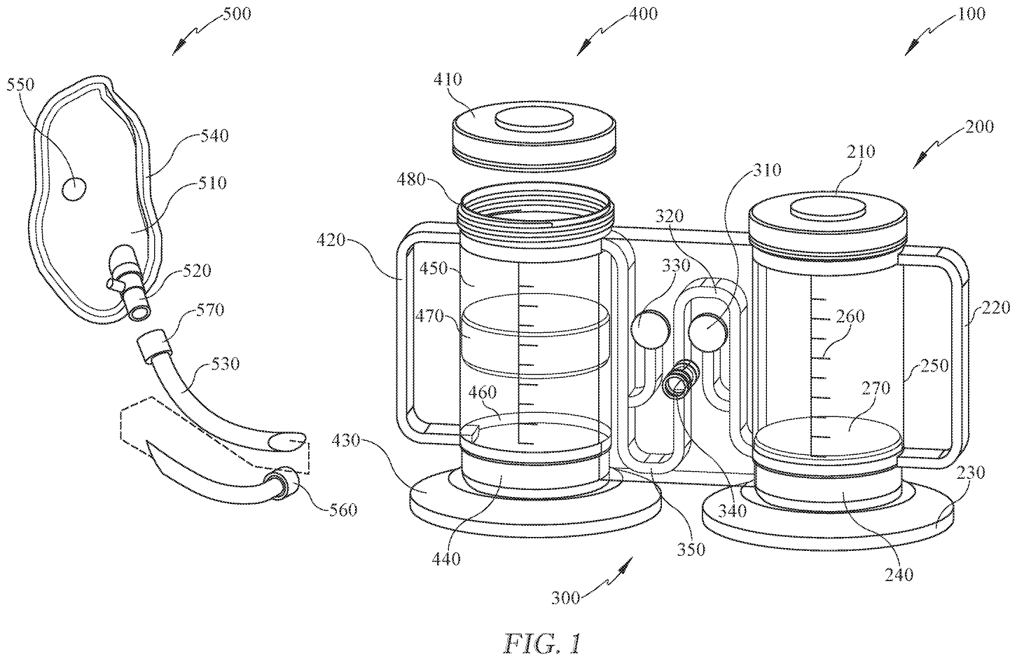

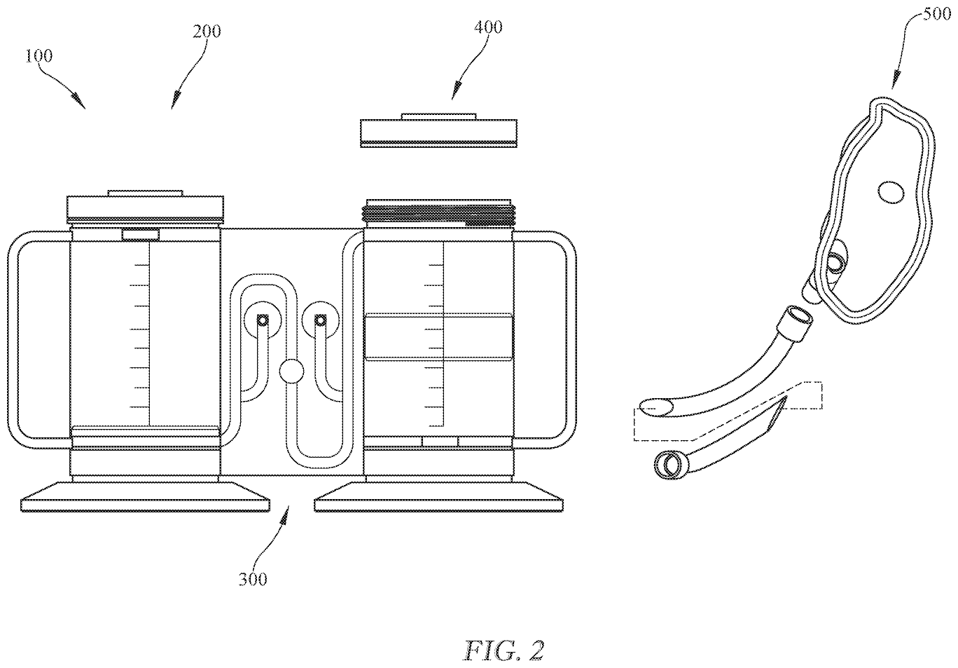

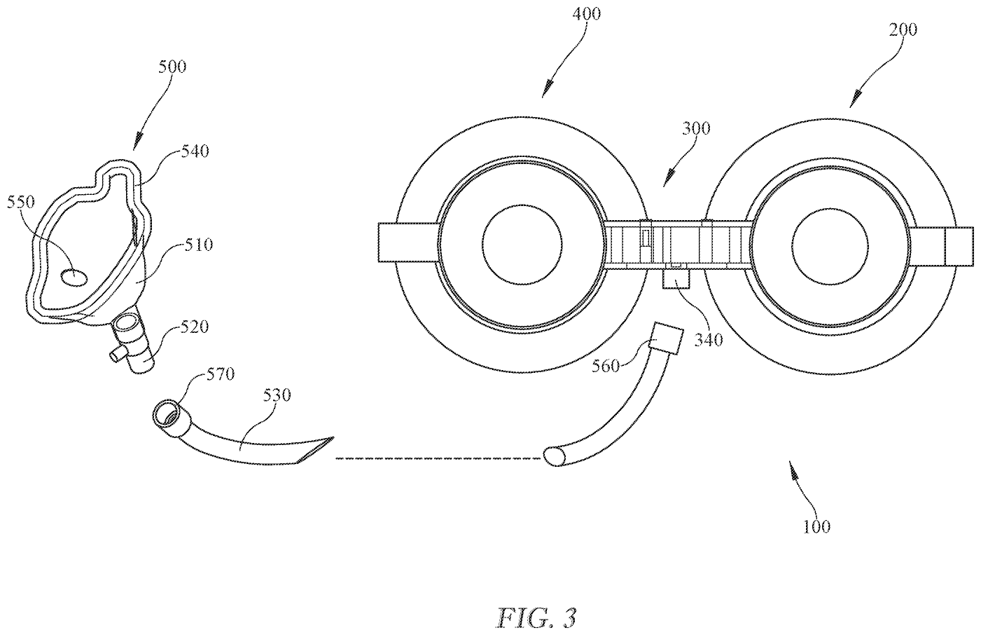

Turning first to FIGS. 1-3, the exhalation side 200 and inhalation side 400 are joined by transfer case 300. The exhalation side 200 comprises cylinder wall 250, which circumscribes piston 270. Cylinder wall 250 tops cylinder floor 240, and is upheld by base 230. Cylinder wall 250 is topped by cylinder cap 210, and in the illustrated embodiment further includes a handle 220. In the illustrated embodiment, cylinder wall 250 is adorned with scale 260. Similarly, the inhalation side 400 comprises cylinder wall 450, which circumscribes piston 470. Cylinder wall 450 tops cylinder floor 440, and is upheld by base 430. Cylinder wall 450 is topped by cylinder cap 410, and in the illustrated embodiment further includes a handle 420. Cylinder cap 410 is affixed to cylinder wall 450 by threads 480. While not shown in the illustrated embodiment, exhalation side 200 has a similar thread configuration to secure cylinder cap 210.

While the illustrated embodiments show cylindrical structures such as cylinder walls 250 and 450, the exhalation side 200 and inhalation side 400 are not limited to a cylindrical construction. In other embodiments, the main body may take the shape of a rectangular prism, triangular prism, or any other prismatic or extruded shapes or have an oval, square, or other cross section shape

The transfer case 300 joins the exhalation side 200 to the inhalation side 400, and comprises a series of channels which terminate in varying branches. In one such branch, on the exhalation side 200, the channels terminate in an exhalation rate meter 310. In another such branch, also on the exhalation side 200, the channel terminates at the cylinder floor 240 of the exhalation side 200. Displaced between a hose inlet 340 and any such termination point on the exhalation side 200 is an exhalation check valve 320. Similarly, in one branch on the inhalation side 400, the channels terminate in an inhalation rate meter 330. In another such branch, also on the inhalation side 400, the channel terminates at the cylinder floor 440 of the inhalation side 400. Displaced between the hose inlet 340 and any such termination point on the inhalation side 400 is an inhalation check valve 350.

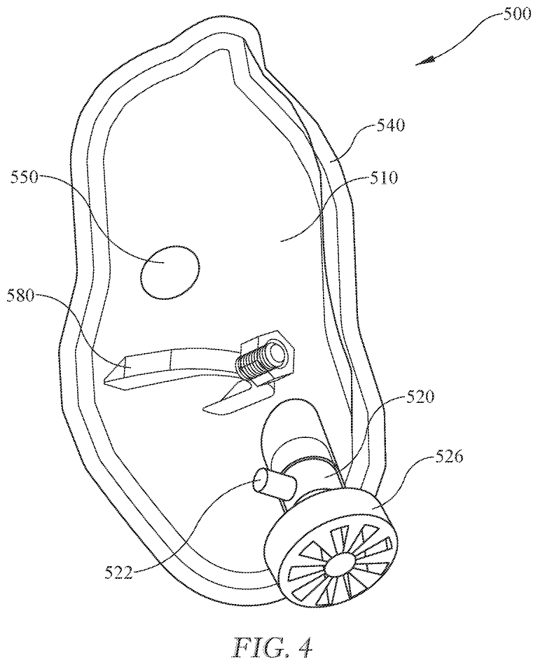

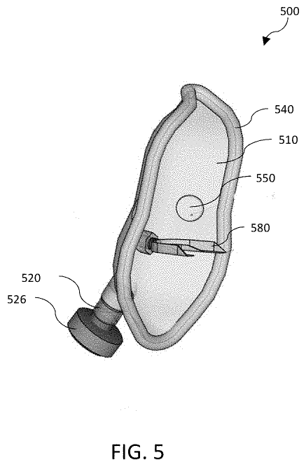

Turning to FIGS. 4-6, a mask assembly 500 is shown according to various embodiments. The embodiments of FIGS. 4 and 5 show a mask face 510, surrounded by a side cushion 540. The mask face 510 further includes a nasal seal 550, formed from a flexible membrane allowing nasal seal 550 to sear an individual nostril at a user's behest. In the illustrated embodiment, mask assembly 500 includes a bite wing 580 for affixing the mask to user's face while the user is still free to breathe through their nose. In other or the same embodiments, the mask can also be fixed with straps, or other methods such as a full-face configuration.

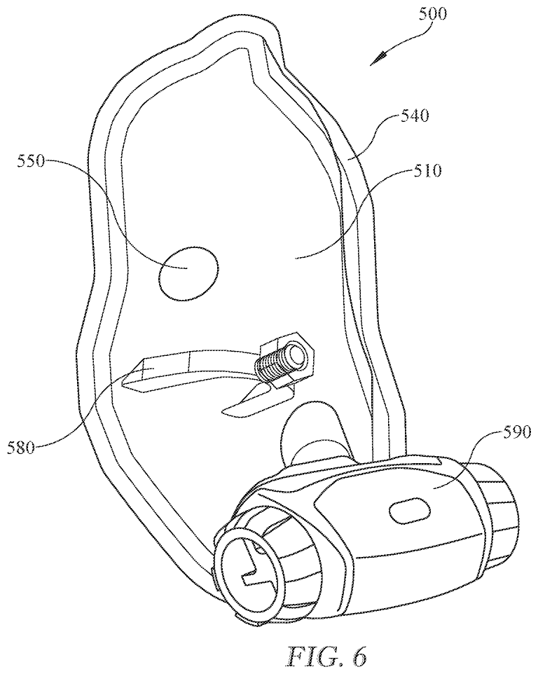

The mask assembly 500 further includes an adapter 520, and in the illustrated embodiment the adapter 520 includes a dial selector 522 for adjusting a restriction within the adapter 520 (not shown). Adapter 520 is configured to connect to many possible attachments, including the hose-mask coupler 570. The hose-mask coupler 570 is connected to a hose which terminates in hose-transfer coupler 560, and is configured to connect to hose inlet 340 in some modes of operation for a breathwork apparatus 100. In other modes, this adapter 520 connects to a particulate filter 526, or a cleaning filter 590 as shown in FIG. 6. In at least some illustrative embodiments, the mask assembly 500 further includes a directional respirator valve capable of cutting off fluid flow from the adapter 520 to either a nosepiece or a mouthpiece, and accordingly diverting fluid flow to the other.

In operation, a breathwork apparatus 100 is configured as a component of a complete breath training system. In at least a first mode of operation, fluid flow through hose inlet 340 is generated by vacuum created in the lungs of a user, which is translated as a negative pressure through mask assembly 500 to the inhalation side 400 through the channels of transfer case 300. This negative pressure allows for inhalation check valve 350 to open, drawing air past inhalation rate meter 330 and applying a vacuum to piston 470. In a reverse mode of operation, fluid flow through hose inlet 340 is generated by positive pressure created in the lungs of a user, holding inhalation check valve 350 closed while opening exhalation check valve 320. As exhaled fluid exits past exhalation rate meter 310, pressure generated caused piston 270 to rise. A user is able to read a scale 260 or 460 to determine the height of the piston on each inhale or exhale mode of operation.

In other operations of a complete breath training system, the mask assembly 500 can independently function with a variety of attachments. In at least illustrative embodiments, the side cushion 540 acts to seal mask assembly 500 to a user's face. In other or the same embodiments, this is crafted from a flexible material such as surgical rubber or similar polymers, or other embodiments wherein the material is slightly breathable. In at least illustrative embodiments of a mask assembly 500, particularly those having a strap configuration, the strap can be removable. In at least illustrative embodiments, a mask assembly 500 is configured such that a user of the mask assembly 500 can only inhale through the nose. In a preferred embodiment of a compact mask assembly 500, which a user can travel with, a mask opening covers only the nasal passageway, such that air may be inhaled through the adapter 520 but can be exhaled freely through the mouth.

In at least some illustrative embodiments of a mask assembly 500, the mask assembly can be configured to attach at adapter 520 to various devices that condition air, such as the particulate filter 526 shown in FIG. 5. However, in other or the same embodiments, other attachments are contemplated, including those which increase concentration in the air of products such as essential oils, concentrates, or herbal materials. In at least some of such embodiments, the mask assembly can humidify incoming air.

In certain exemplary embodiments, the mask assembly 500 further includes a sensor assembly that includes a pressure or fluid velocity sensor. In other or the same embodiments, sensor assembly can signal through audio, visual, digital transmission, or the like, that a particular pressure threshold has been exceeded. The sensor assembly can further include an oxygen sensor, carbon dioxide sensor, carbon monoxide sensor, moisture content sensor, or spectrometer.

According to at least some modes of use, the sensor assembly can connect to a computer or mobile device to transfer sensor data to a mobile application. In at least some illustrative embodiments, the number of pistons 270 and 470 and/or amount of mass represented by the totality of a multiplicity of pistons 270 and 470 can be read by sensors in the sensor array and transferred to the mobile application. In at least some illustrative embodiments, the mobile application can record analytical data about a user's experience with the breath training system, including the duration of a user's use session of a breathwork apparatus, the amount of mass represented by pistons 270 and 470, the velocity of air inhaled by the user, the amount of air inhaled by the user, and the elemental content of the air inhaled by the user, along with storing these data points in a tracking application.

In at least some illustrative embodiments, the sensor array and corresponding mobile application can monitor and/or calculate parameters such as volume of inspired and expired breath, rate of inspired and expired breath, air speed of inspired and expired breath, rate of oxygen use by the user, oxygen saturation levels experienced by the user, instant heart rate, heart rate variability, and minute ventilation.

According to at least some modes of use, a user can increase the mass reflected by pistons 270 and 470 while maintaining a target air velocity through the sensor array in order to strengthen the diaphragm of the user. In at least such embodiments, carious time, mass, and flow parameters captured by sensor array are accordingly logged into a mobile application.

According to at least some illustrative embodiments, the mask assembly can be configured to allow a user to inhale only through the nose, such that the breath training device promotes controlled airflow through the nasal passages and into the lungs.

According to at least some illustrative embodiments, a breathwork apparatus 100 further contains a belt device, wherein the belt device measures the expansion of the abdomen and/or rib cage of a user to determine whether or not the user is properly activating their diaphragm when using a breath training system. According to at least some of such embodiments, this belt device further includes a pressure diode or strain gauge to connect to the mobile application, wherein the mobile application can use the diode location and pressure to visually show the user how their diaphragm is being utilized, and advising the user on how to improve their diaphragm control to achieve a desired result.

In at least some illustrative embodiments, the sensor array and mobile application combine to act as a training apparatus for musicians. In other or the same embodiments, the adapter 520 can affix to a device having an orifice where a user can audibly hear their inhaled an exhaled breath. In at least some of these embodiments, the user can utilize a metronome within the mobile application to better time their breath, and can further utilize a chromatic tuner contained within the mobile application, receiving pitch information from a microphone in the sensor array, the train the user's breath in accordance with a target pitch.

Although only a number of exemplary embodiments have been described in detail above, those skilled in the art will readily appreciate that many modifications are possible in the exemplary embodiments without materially departing from the novel teachings and advantages. Accordingly, all such modifications are intended to be included within the scope of this disclosure as defined in the following claims.

While the methods, equipment and systems have been described in connection with specific embodiments, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect.

As used in the specification and the appended claims the singular forms “a,” “an”, and “the” include plural referents unless the context clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods, equipment and systems. These and other components are disclosed herein, and it is understood that when combinations, subsets, interactions, groups, etc., of these components are disclosed that while specific reference of each various individual and collective combinations and permutation of these may not be explicitly disclosed, each is specifically contemplated and described herein, for all methods, equipment and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

It should further be noted that any patents, applications and publications referred to herein are incorporated by reference in their entirety.

What is claimed is:

1. A breathwork apparatus, comprising:
(a) an exhalation side;
(b) a transfer case;
(c) an inhalation side; and,
(d) a mask assembly,
wherein the exhalation side and inhalation side are joined by transfer case which comprises a plurality of channels, such that at least a first channel of a plurality of channels terminates in an exhalation rate meter, and at least a second channel of the plurality of channels terminates in an inhalation rate meter.

2. The breathwork apparatus of claim 1, wherein the exhalation side comprises an exhalation check valve.

3. The breathwork apparatus of claim 1, wherein the inhalation side comprises an inhalation check valve.

4. The breathwork apparatus of claim 1, wherein the inhalation side comprises a base, a cylinder floor, a cylinder wall, and a piston.

5. The breathwork apparatus of claim 1, wherein the exhalation side comprises a base, a cylinder floor, a cylinder wall, and a piston.

6. The breathwork apparatus of claim 1, wherein the mask assembly comprises a mask connection in the form of a hose.

7. The breathwork apparatus of claim 1, wherein the mask assembly comprises a particulate filter.

8. The breathwork apparatus of claim 1, wherein the mask assembly comprises a nasal seal.

9. The breathwork apparatus of claim 1, wherein the mask assembly comprises an adapter with a dial selector, wherein the dial selector adjusts a restriction within the adapter.

10. A breath training system, comprising:
(a) a breathwork apparatus; and.
(b) a mask assembly,
wherein the breathwork apparatus has an exhalation side, an inhalation side, and a transfer case between the inhalation side and the exhalation side,
wherein the mask assembly is selectively attachable to the breathwork apparatus, and,
wherein the mask assembly includes a nasal seal, such that when the mask assembly is detached from the breathwork apparatus, an operator of the mask assembly cannot exhale through the operator's nose.

11. The breath training system of claim 10, wherein the mask assembly comprises an adapter, the adapter operable to connect the mask assembly to the breathwork apparatus, and alternative to connect the mask assembly to a device that conditions air.

12. The breath training system of claim 11, wherein the operator of the mask assembly inhales through the device that conditions air when the operator inhales through the operator's nose.

13. The breath training system of claim 11, wherein the mask assembly includes a sensor assembly, wherein the sensor assembly comprises sensors selected from the group consisting of pressure sensors, fluid velocity sensors, oxygen sensors, carbon dioxide sensors, carbon monoxide sensors, moisture content sensors, and spectrometers.

14. The breath training system of claim 13, wherein the sensor array transmits data from at least one sensor in the sensor array to a mobile application.

15. The breath training system of claim 14, wherein the mobile application monitors data to calculate a parameter selected from the group consisting of volume of operator's inspired and expired breath, rate of operator's inspired and expired breath, air speed of operator's inspired and expired breath, rate of oxygen use by the operator, oxygen saturation levels experienced by the operator, instant heart rate of the operator, heart rate variability of the operator, and minute ventilation.

16. The breath training system of claim 10, further comprising a belt device measuring the expansion of the abdomen or rib cage of an operator during operation of the system.

17. A method for operating a breath training system, the method comprising:
(a) providing a breathwork apparatus having an exhalation side, a transfer case, and an inhalation side;
(b) generating fluid flow, by vacuum produced in the lungs of an operator, through a hose inlet in the transfer case;
(c) generating a negative pressure in the inhalation side through the transfer case, the negative pressure opening a check valve in the inhalation side;
(d) applying vacuum to a piston in the inhalation side; and,
(e) reading a scale to determine the motion of the piston within the inhalation side.

18. The method of claim 17, wherein the vacuum produced in the lungs of the operator is transferred through a mask assembly.

19. The method of claim 18, wherein the mask assembly has a nasal seal such that the vacuum generated in the operator's lungs is transferred through the operator's nose.

20. The method of claim 19, wherein the mask assembly is configured to allow the operator to exhale through the mouth.
